(19) United States
(12) Patent Application Publication
(10) Pub. No.: US 2026/0272400 A1
(43) Pub. Date: Sep. 17, 2026

(54) ULTRASOUND DIAGNOSTIC APPARATUS AND METHOD OF CONTROLLING ULTRASOUND DIAGNOSTIC APPARATUS

(71) Applicant: FUJIFILM Corporation, Tokyo (JP)

(72) Inventor: Kaito TAKASHIMA, Kanagawa (JP)

(73) Assignee: FUJIFILM Corporation, Tokyo (JP)

(21) Appl. No.: 19/560,148

(22) Filed: Mar. 9, 2026

(30) Foreign Application Priority Data
Mar. 12, 2025 (JP) 2025-039199

(51) Int. Cl. A61B 8/06; A61B 8/00; A61B 8/08

(52) U.S. Cl. CPC A61B 8/06; A61B 8/0883; A61B 8/461; A61B 8/488

(57) ABSTRACT

Provided are an ultrasound diagnostic apparatus and a control method of an ultrasound diagnostic apparatus that can accurately measure a path length of a left ventricular outflow tract and a velocity-time integral value of blood flow in the left ventricular outflow tract.

An ultrasound diagnostic apparatus includes: an image recognition unit (25) configured to recognize, from the ultrasound image acquired by the image acquisition unit, a left ventricular outflow tract depicted in the cross section; a first measurement unit (26) configured to acquire, as a first measurement value, a path length of the left ventricular outflow tract recognized by the image recognition unit; a gate setting unit (27) configured to set a Doppler gate in the left ventricular outflow tract recognized by the image recognition unit; a second measurement unit (28) configured to acquire, as a second measurement value, a velocity time integral value of blood flow in the left ventricular outflow tract by measuring a blood flow velocity based on a Doppler frequency shift at a position of the Doppler gate set by the gate setting unit; a determination unit (29) configured to determine whether the first measurement value acquired by the first measurement unit and the second measurement value acquired by the second measurement unit are respectively within a predetermined first allowable range and a predetermined second allowable range; and a notification unit (30) configured to notify a user of a determination result by the determination unit (29).

### CROSS-REFERENCE TO RELATED APPLICATIONS

The present application claims priority under 35 U.S.C. § 119 to Japanese Patent Application No. 2025-039199, filed on Mar. 12, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method for the ultrasound diagnostic apparatus.

### 2. Description of the Related Art

In the related art, a so-called cardiac output is calculated by capturing an ultrasound image representing a tomographic plane of a heart of a subject using a so-called ultrasound diagnostic apparatus and analyzing the captured ultrasound image. The calculation of the cardiac output is often performed by calculation steps of (1) measuring a diameter of a left ventricular outflow tract in an ultrasound image representing a so-called parasternal left ventricular long-axis tomographic plane of the heart in a middle systolic phase, and calculating a cross-sectional area of the left ventricular outflow tract, (2) calculating a velocity-time integral value of blood flow in the left ventricular outflow tract using a so-called pulse Doppler method by using an ultrasound image representing a so-called apical five-chamber tomographic plane or a so-called apical three-chamber tomographic plane, (3) calculating a so-called stroke volume by multiplying the cross-sectional area of the left ventricular outflow tract by the velocity-time integral value of the blood flow in the left ventricular outflow tract, and (4) calculating the cardiac output by multiplying the stroke volume by a heart rate.

As described above, in a case of measuring the stroke volume or the cardiac output, two tomographic planes of the heart are often imaged, which causes a large amount of time to be required for the measurement. Therefore, for example, as disclosed in JP2020-075104A, the stroke volume or the cardiac output can be calculated by calculating both the diameter of the left ventricular outflow tract and the velocity-time integral value of the blood flow in the left ventricular outflow tract by using only an ultrasound image representing the parasternal left ventricular long-axis tomographic plane representing one type of tomographic plane or using only an ultrasound image representing the apical five-chamber tomographic plane.

### SUMMARY OF THE INVENTION

However, in a case in which the technique of JP2020-075104A is used, it is necessary for a user such as a doctor to manually adjust a position or a posture of an ultrasound probe for displaying an appropriate tomographic plane, to dispose a measurement line for measuring the diameter of the left ventricular outflow tract in the ultrasound image, and to dispose a so-called Doppler gate for measuring the velocity-time integral value of the blood flow in the left ventricular outflow tract in the ultrasound image. Therefore, a measurement error may occur in the diameter of the left ventricular outflow tract and the velocity-time integral value of the blood flow in the left ventricular outflow tract. In a case in which the measurement error occurs as described above, the stroke volume and the cardiac output may not be accurately calculated.

The present invention has been made to solve such problems in the related art, and an object of the present invention is to provide an ultrasound diagnostic apparatus and a control method of an ultrasound diagnostic apparatus that can accurately measure a path length of a left ventricular outflow tract and a velocity-time integral value of blood flow in the left ventricular outflow tract.

With the following configurations, the above-described object can be achieved.
[1] An ultrasound diagnostic apparatus comprising: an image acquisition unit that acquires an ultrasound image in which a predetermined tomographic plane of a heart of a subject is imaged; an image recognition unit that recognizes a left ventricular outflow tract displayed on the tomographic plane from the ultrasound image acquired by the image acquisition unit; a first measurement unit that acquires a path length of the left ventricular outflow tract recognized by the image recognition unit as a first measurement value; a gate setting unit that sets a Doppler gate on the left ventricular outflow tract recognized by the image recognition unit; a second measurement unit that acquires a velocity-time integral value of blood flow in the left ventricular outflow tract as a second measurement value by measuring a blood flow velocity based on a Doppler frequency shift at a position of the Doppler gate set by the gate setting unit; a determination unit that determines whether or not the first measurement value acquired by the first measurement unit and the second measurement value acquired by the second measurement unit are within a predetermined first allowable range and a predetermined second allowable range, respectively; and a notification unit that notifies a user of a determination result of the determination unit.
[2] The ultrasound diagnostic apparatus according to [1], in which the image acquisition unit acquires an ultrasound image in which a parasternal left ventricular long-axis tomographic plane is imaged as the predetermined tomographic plane.
[3] The ultrasound diagnostic apparatus according to [2], in which the notification unit is configured to: notify a proposal to reacquire the ultrasound image for the parasternal longitudinal axis section of the left ventricle in a case where the determination unit determines that the first measurement value is out of the first allowable range; and notify a proposal to reacquire the ultrasound image using, as the predetermined cross section, an apical three-chamber section or an apical five-chamber section in a case where the determination unit determines that the second measurement value is out of the second allowable range.
[4] The ultrasound diagnostic apparatus according to [1], in which the image acquisition unit is configured to acquire the ultrasound image in which, as the predetermined cross section, an apical three-chamber section is imaged.
[5] The ultrasound diagnostic apparatus according to [4], in which the notification unit is configured to: notify a proposal to reacquire the ultrasound image using, as the predetermined cross section, the parasternal longitudinal axis section of the left ventricle in a case where the determination unit determines that the first measured value is out of the first allowable range; and notify a proposal to reacquire the ultrasound image, as the predetermined cross section, an apical three-chamber section or notify a proposal to reacquire the ultrasound image using, as the predetermined cross section, an apical five-chamber section in a case where the determination unit determines that the second measurement value is out of the second allowable range.
[6] The ultrasound diagnostic apparatus according to [1], in which the image acquisition unit is configured to acquire the ultrasound image in which, as the predetermined cross section, an apical five-chamber section is imaged.
[7] The ultrasound diagnostic apparatus according to [6], in which the notification unit is configured to: notify a proposal to reacquire the ultrasound image using, as the predetermined cross section, the parasternal longitudinal axis section of the left ventricle in a case where the determination unit determines that the first measurement value is out of the first allowable range; and notify a proposal to reacquire the ultrasound image, as the predetermined cross section, an apical five-chamber section or notify a proposal to reacquire the ultrasound image using, as the predetermined cross section, an apical three-chamber section in a case where the determination unit determines that the second measurement value is out of the second allowable range.
[8] The ultrasound diagnostic apparatus according to any one of [1] to [7], further comprising: a monitor, in which the notification unit is configured to display the determination result by the determination unit on the monitor.
[9] The ultrasound diagnostic apparatus according to any one of [1] to [7], further comprising: a speaker, in which the notification unit is configured to output the determination result by the determination unit as sound from the speaker.
[10] The ultrasound diagnostic apparatus according to any one of [1] to [9], in which the determination unit is further configured to determine that the first measurement value is out of the first allowable range in a case where an angle formed between a measurement line used by the first measurement unit in measuring the path length of the left ventricular outflow tract as the first measurement value and a scan line passing through a center point of the Doppler gate is within a predetermined angle range.
[11] The ultrasound diagnostic apparatus according to any one of [1] to [9], in which the second measurement unit is configured to correct the blood flow velocity based on an angle formed between a measurement line used by the first measurement unit in measuring the path length of the left ventricular outflow tract as the first measurement value and a scan line passing through a center point of the Doppler gate, and calculate the velocity time integral value using the corrected blood flow velocity.
[12] A control method of an ultrasound diagnostic apparatus, the method comprising: acquiring an ultrasound image in which a predetermined cross section of a heart of a subject is imaged; recognizing, from the acquired ultrasound image, a left ventricular outflow tract depicted in the cross section; acquiring, as a first measurement value, a path length of the recognized left ventricular outflow tract; setting a Doppler gate in the recognized left ventricular outflow tract; acquiring, as a second measurement value, a velocity time integral value of blood flow in the left ventricular outflow tract by measuring a blood flow velocity based on a Doppler frequency shift at a position of the set Doppler gate; determining whether the acquired first measurement value and the acquired second measurement value are respectively within a predetermined first allowable range and a predetermined second allowable range; and notifying a user of a determination result.

In the ultrasound diagnostic apparatus according to the present invention, the ultrasound diagnostic apparatus includes an image acquisition unit configured to acquire an ultrasound image in which a predetermined cross section of a heart of a subject is imaged; an image recognition unit configured to recognize, from the ultrasound image acquired by the image acquisition unit, a left ventricular outflow tract depicted in the cross section; a first measurement unit configured to acquire, as a first measurement value, a path length of the left ventricular outflow tract recognized by the image recognition unit; a gate setting unit configured to set a Doppler gate in the left ventricular outflow tract recognized by the image recognition unit; a second measurement unit configured to acquire, as a second measurement value, a velocity time integral value of blood flow in the left ventricular outflow tract by measuring a blood flow velocity based on a Doppler frequency shift at a position of the Doppler gate set by the gate setting unit; a determination unit configured to determine whether the first measurement value acquired by the first measurement unit and the second measurement value acquired by the second measurement unit are respectively within a predetermined first allowable range and a predetermined second allowable range; and a notification unit configured to notify a user of a determination result by the determination unit.

### BRIEF DESCRIPTION OF THE DRAWINGS

FIG. 1 is a block diagram showing a configuration of an ultrasound diagnostic apparatus according to an embodiment of the present invention.

FIG. 2 is a block diagram showing an internal configuration of a transmission and reception circuit in an embodiment of the present invention.

FIG. 3 is a block diagram showing an internal configuration of an image generation unit in an embodiment of the present invention.

FIG. 4 is a diagram showing an example of an ultrasound image representing a parasternal left ventricular long-axis tomographic plane.

FIG. 5 is a diagram showing an example of an ultrasound image representing an apical five-chamber tomographic plane.

FIG. 6 is a diagram showing an example of an ultrasound image representing an apical three-chamber tomographic plane.

FIG. 7 is a diagram showing an example of a measurement line disposed in the ultrasound image representing the parasternal left ventricular long-axis tomographic plane.

FIG. 8 is a diagram showing an example of a Doppler gate disposed in the ultrasound image representing the parasternal left ventricular long-axis tomographic plane.

FIG. 9 is a block diagram showing an internal configuration of a second measurement unit in the embodiment of the present invention.

FIG. 10 is a diagram showing an example of a determination result of a path length of a left ventricular outflow tract displayed on a monitor.

FIG. 11 is a flowchart showing an operation of the ultrasound diagnostic apparatus according to an embodiment of the present invention.

FIG. 12 is a block diagram showing a partial configuration of a modification example of the ultrasound diagnostic apparatus according to the embodiment of the present invention.

FIG. 13 is a diagram showing an angle between a scanning line passing through a center of a Doppler gate disposed in the ultrasound image representing the parasternal left ventricular long-axis tomographic plane and a measurement line.

### DESCRIPTION OF THE PREFERRED EMBODIMENTS

Hereinafter, embodiments of the present invention will be described based on the accompanying drawings.

The following configuration requirements are described based on a representative embodiment of the present invention, but the present invention is not limited to such an embodiment.

It should be noted that, in the present specification, a numerical range represented by “to” means a range including numerical values described before and after “to”, both ends inclusive, as a lower limit value and an upper limit value.

In the present specification, “the same” includes an error range generally allowed in the technical field.

### Embodiment

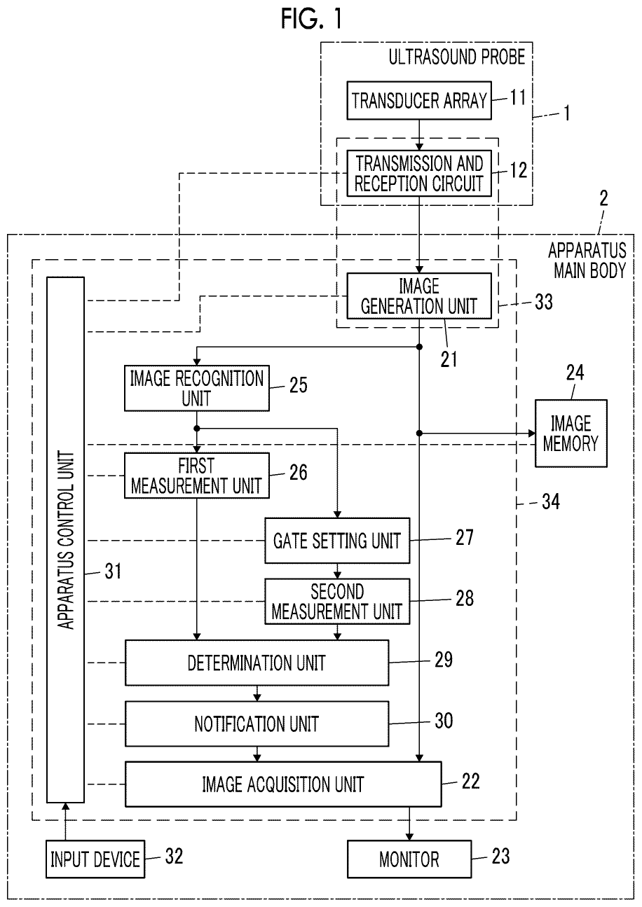

FIG. 1 shows a configuration of an ultrasound diagnostic apparatus according to an embodiment of the present invention. The ultrasound diagnostic apparatus comprises an ultrasound probe 1 and an apparatus main body 2 connected to each other by so-called wired communication or so-called wireless communication.

The ultrasound probe 1 comprises a transducer array 11 and a transmission and reception circuit 12 connected to the transducer array 11.

The apparatus main body 2 comprises an image generation unit 21 connected to the transmission and reception circuit 12. In the apparatus main body 2, a display control unit 22 and a monitor 23 are sequentially connected to the image generation unit 21. In addition, an image memory 24 and an image recognition unit 25 are connected to the image generation unit 21. A first measurement unit 26 and a gate setting unit 27 are connected to the image recognition unit 25. A second measurement unit 28 is connected to the gate setting unit 27. A determination unit 29 is connected to the first measurement unit 26 and the second measurement unit 28. A notification unit 30 is connected to the determination unit 29. The notification unit 30 is connected to the display control unit 22. In addition, an apparatus control unit 31 is connected to the transmission and reception circuit 12, the image generation unit 21, the image memory 24, the image recognition unit 25, the first measurement unit 26, the gate setting unit 27, the second measurement unit 28, the determination unit 29, and the notification unit 30. An input device 31 is connected to the input device 32.

The transmission and reception circuit 12 and the image generation unit 21 constitute an image acquisition unit 33. In addition, a processor 34 for the apparatus body 2 is configured by the image generation unit 21, the display control unit 22, the image recognition unit 25, the first measurement unit 26, the gate setting unit 27, the second measurement unit 28, the determination unit 29, the notification unit 30, and the apparatus control unit 31.

The transducer array 11 of the ultrasound probe 1 has a plurality of ultrasonic transducers arranged in a one-dimensional or two-dimensional manner. According to a drive signal supplied from the transmission and reception circuit 12, each of the ultrasonic transducers transmits an ultrasonic wave and receives an ultrasound echo from the subject to output a signal based on the ultrasound echo. For example, each ultrasound transducer is configured by forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 33, which is composed of the transmission/reception circuit 12 and the image generation unit 21, acquires ultrasound images of a plurality of frames as a moving image in which a heart of the subject is imaged, by transmitting and receiving ultrasound beams using the ultrasound probe 1.

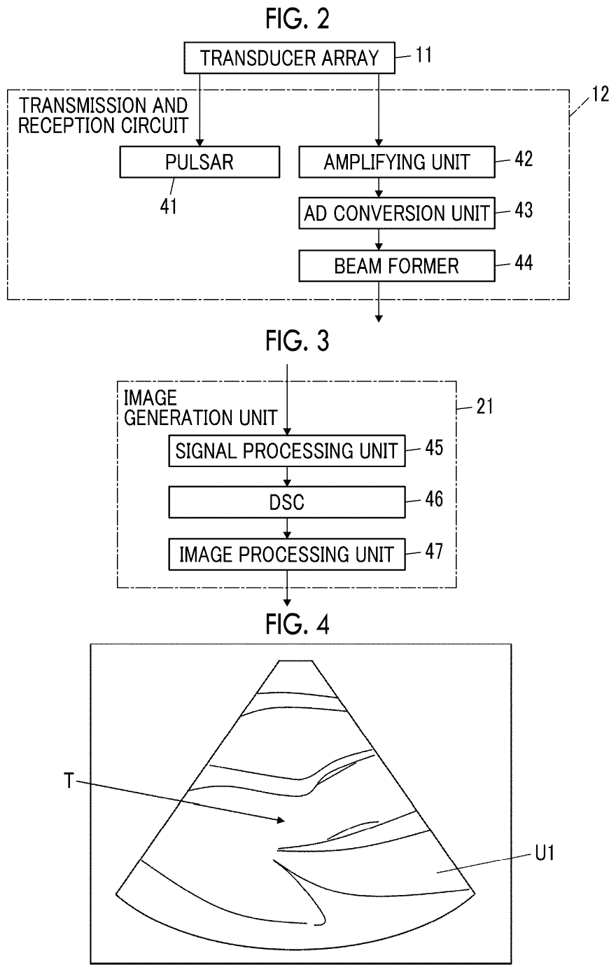

The transmission and reception circuit 12 transmits the ultrasound waves from the transducer array 11 and generates a sound ray signal based on reception signals acquired by the transducer array 11 under control of the apparatus control unit 31. As shown in FIG. 2, the transmission and reception circuit 12 includes a pulsar 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus control unit 31 so that the ultrasound waves transmitted from the plurality of ultrasound oscillators of the transducer array 11 form an ultrasound beam, and supplies each drive signal to the plurality of ultrasound oscillators. In this way, in a case where a pulsed or continuous wave-like voltage is applied to the electrodes of the ultrasound transducer of the transducer array 11, the piezoelectric body expands and contracts to generate a pulsed or continuous wave-like ultrasound wave from each of the ultrasound transducers, whereby an ultrasound beam is formed from the combined wave of these ultrasound waves.

The transmitted ultrasound beam is, for example, reflected in a target such as a part of the subject and propagates toward the transducer array 11 of the ultrasound probe 1. The ultrasound echo propagating toward the transducer array 11 in this way is received by each of the ultrasound transducers constituting the transducer array 11. In this case, each of the ultrasound transducers constituting the transducer array 11 receives the propagating ultrasound echo to expand and contract to generate the reception signal, which is an electrical signal, and outputs these reception signals to the amplifying unit 42.

The amplifying unit 42 amplifies the signal input from each of the ultrasound transducers constituting the transducer array 11 and transmits the amplified signal to the AD conversion unit 43. The AD conversion unit 43 converts the signal transmitted from the amplifying unit 42 into digital reception data. The beam former 44 performs so-called reception focus processing by applying and adding the delay to each reception data received from the AD conversion unit 43. By this reception focus processing, each reception data converted by the AD conversion unit 43 is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series to each other.

The signal processing unit 45 corrects attenuation by distance of the sound ray signal received from the transmission and reception circuit 12 in accordance with depths of reflection positions of the ultrasound waves using a sound speed value set by the apparatus control unit 31 and then performs envelope detection processing on the sound ray signal to generate a B-mode image signal that is tomographic image information related to tissues inside the subject.

The DSC 46 converts (raster-converts) the B-mode image signal generated by the signal processing unit 45 into the image signal in accordance with a normal television signal scanning method.

The image processing unit 47 performs various kinds of necessary image processing such as gradation processing on the B-mode image signal input from the DSC 46, and then sends the B-mode image signal to the display control unit 22, the image memory 24, and the image recognition unit 25. Hereinafter, the B-mode image signal that is image-processed by the image processing unit 47 will be referred to as an ultrasound image.

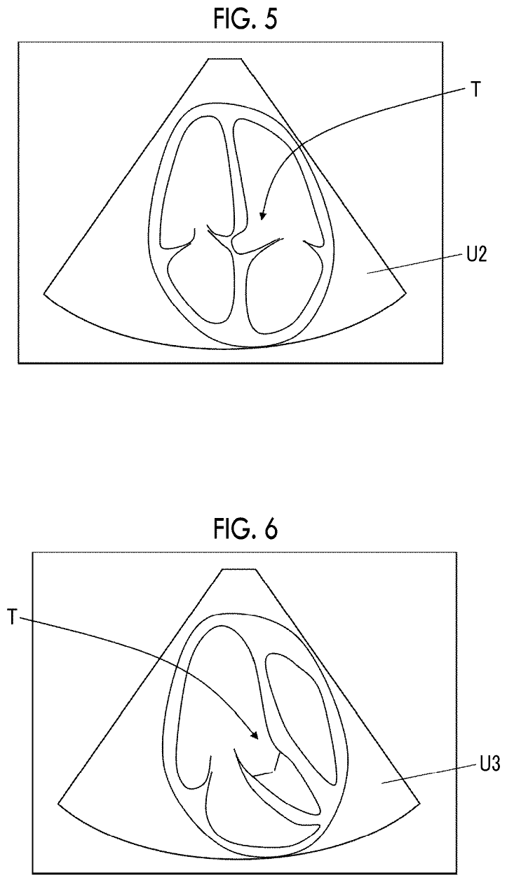

In the present invention, the image acquisition unit 33 acquires an ultrasound image in which a predetermined tomographic plane of the heart of the subject is imaged. For example, as schematically shown in FIG. 4, the image acquisition unit 33 can acquire an ultrasound image representing a so-called parasternal left ventricular long-axis tomographic plane. The parasternal left ventricular long-axis tomographic plane is a tomographic plane including a left ventricle, a left ventricular outflow tract T, a left atrium, and a right ventricle. In addition, for example, as schematically shown in FIG. 5, the image acquisition unit 33 can acquire an ultrasound image U2 representing an apical five-chamber tomographic plane. The apical five-chamber tomographic plane is a tomographic plane of the heart including five lumens of the left ventricle, the left ventricular outflow tract T, the left atrium, the right ventricle, and the right atrium. In addition, for example, as schematically shown in FIG. 6, the image acquisition unit 33 can acquire an ultrasound image U3 representing an apical three-chamber tomographic plane. The apical three-chamber tomographic plane is a tomographic plane of the heart including three lumens of the left ventricle, the left atrium, and the right ventricle. Both the apical five-chamber tomographic plane and the apical three-chamber tomographic plane include the left ventricular outflow tract T.

The image memory 24 is a memory that stores the ultrasound image acquired by an image acquisition unit 33. The user can use the ultrasound image stored in the image memory 24, for example, to check the ultrasound image after the examination. Here, as the image memory 24, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used.

The image recognition unit 25 recognizes the left ventricular outflow tract T displayed on the predetermined tomographic plane from the ultrasound image acquired by the image acquisition unit 33. The image recognition unit 25 can recognize the left ventricular outflow tract T in the parasternal left ventricular long-axis tomographic plane, the apical five-chamber tomographic plane, and the apical three-chamber tomographic plane by using, for example, a so-called template matching method of searching the ultrasound image using template data representing a typical image pattern of the left ventricular outflow tract T in the parasternal left ventricular long-axis tomographic plane, the apical five-chamber tomographic plane, and the apical three-chamber tomographic plane, the template data being stored in advance. The image recognition unit 25 has, for example, a trained model in so-called machine learning in which the image pattern of the left ventricular outflow tract T in the parasternal left ventricular long-axis tomographic plane, the apical five-chamber tomographic plane, and the apical three-chamber tomographic plane is to be learned, and can also output a recognition result to the trained model by inputting the ultrasound image to the trained model.

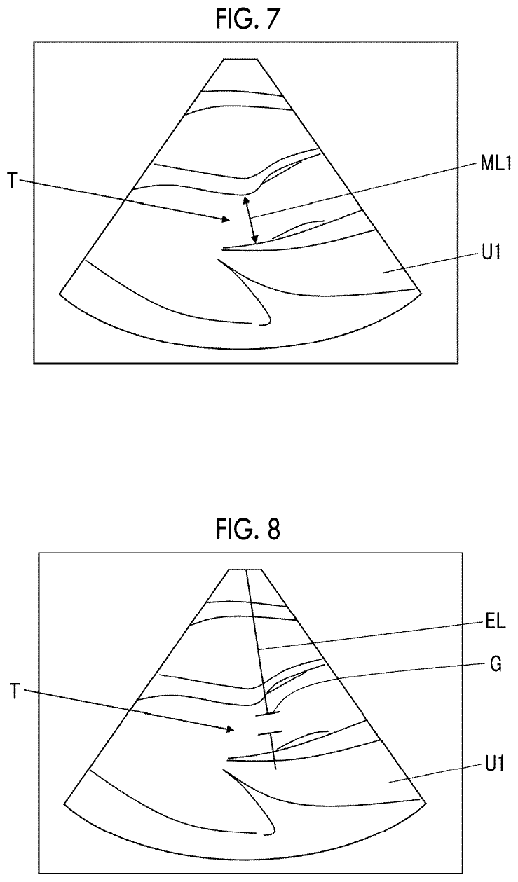

The first measurement unit 26 acquires a path length of the left ventricular outflow tract T recognized by the image recognition unit 25, that is, the left ventricular outflow tract, as the first measurement value. The first measurement unit 26 can measure, for example, as shown in FIG. 7, a path length of a measurement line ML1 disposed on the left ventricular outflow tract T as the left ventricular outflow tract. The first measurement unit 26 can also dispose the measurement line ML1 at a position designated by the user on the ultrasound image U1 via the input device 32.

The gate setting unit 27 sets a so-called Doppler gate G on the left ventricular outflow tract T recognized by the image recognition unit 25, for example, as shown in FIG. 8. The Doppler gate G is used to measure the blood flow velocity in the Doppler gate G by a so-called pulse Doppler method. A scanning line EL passing through the center of the Doppler gate G is associated with the Doppler gate G. The gate setting unit 27 can also set the Doppler gate G at a position designated by the user via the input device 32.

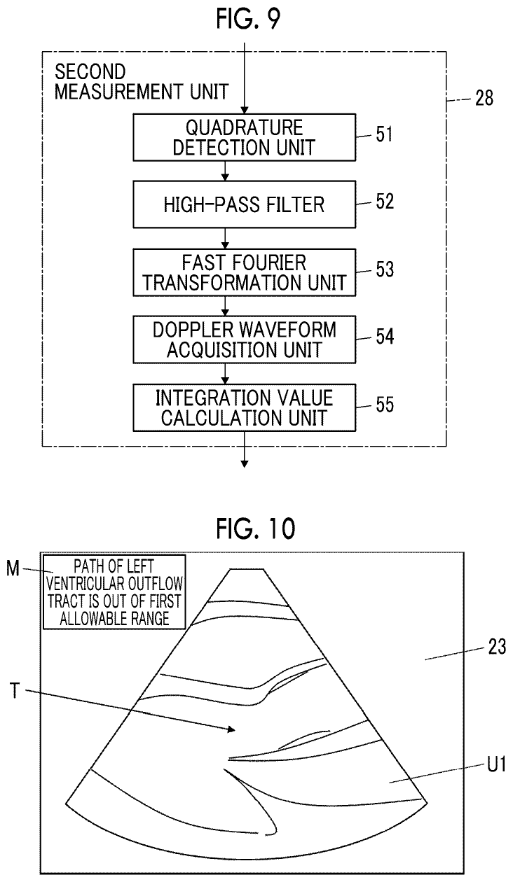

The second measurement unit 28 acquires a velocity-time integral value of blood flow, that is, the left ventricular outflow tract blood flow in the left ventricular outflow tract T as the second measurement value by measuring the blood flow velocity based on the Doppler frequency shift at the position of the Doppler gate G set by the gate setting unit 27. As shown in FIG. 9, the second measurement unit 28 has a configuration in which an orthogonal detection unit 51, a high-pass filter 52, a fast Fourier transform unit 53, a Doppler waveform acquisition unit 54, and an integral value calculation unit 55 are connected in series.

The orthogonal detection unit 51 mixes the acoustic signal received from the transceiver circuit 12 with a carrier signal of a reference frequency to orthogonally detect the acoustic signal and convert the acoustic signal into a complex signal.

The high-pass filter 52 functions as a so-called wall filter, and removes a frequency component derived from the motion of the body tissue inside the subject, from the complex data generated by the quadrature detection unit 51.

The fast Fourier transformer 53 performs a Fourier transform on the complex data of a plurality of sample points to perform frequency analysis, obtains the blood flow velocity, and generates a spectrum signal.

The Doppler waveform acquisition unit 54 acquires a Doppler waveform image signal by representing the magnitude of each frequency component with brightness while aligning the spectrum signal generated by the fast Fourier transform unit 53 on a time axis. The Doppler waveform image signal is called a so-called Doppler image, represents a time-varying waveform of the blood flow velocity, shows a time axis on a horizontal axis, shows a Doppler frequency shift, that is, a flow velocity on a vertical axis, and shows power at each frequency component with brightness of the waveform.

The integral value calculation unit 55 specifies a cardiac cycle period in the Doppler waveform image signal, and calculates the velocity-time integral value by time-integrating the value of the flow velocity in the Doppler waveform image signal for the cardiac cycle period. The integral value calculation unit 55 can specify a repetition unit of the waveform in the Doppler waveform image signal by analyzing the Doppler waveform image signal, and can specify a period to which the specified repetition unit belongs as the cardiac cycle period.

Since the Doppler gate G is set on the left ventricular outflow tract T on the ultrasound image U2 by the gate setting unit 27, the velocity-time integral value calculated by the integral value calculation unit 55 in this way is a value obtained by integrating the velocity of the blood flow flowing through the left ventricular outflow tract T for the cardiac cycle period. The velocity-time integral value can be used to calculate a so-called stroke volume and a cardiac output together with the path length of the left ventricular outflow tract measured in the ultrasound image representing the so-called parasternal left ventricular long-axis tomographic plane or the like.

The processing of calculating the velocity-time integral value by the orthogonal detection unit 51, the high-pass filter 52, the fast Fourier transform unit 53, the Doppler waveform acquisition unit 54, and the integral value calculation unit 55 is performed after the transducer array 11 is driven by the drive signal to transmit the ultrasonic wave in a pulsed manner under the control of the transceiver circuit 12 and the apparatus control unit 31.

The left ventricular outflow tract measured by the first measurement unit 26 and the velocity-time integral value of the left ventricular outflow tract blood flow measured by the second measurement unit 28 are used to calculate a so-called stroke volume and a cardiac output. Specifically, a cross-sectional area of the left ventricular outflow tract T can be calculated from the path length of the left ventricular outflow tract by approximating a cross-sectional shape perpendicular to a blood flow direction of the left ventricular outflow tract T to a circle, and the stroke volume can be calculated by multiplying the calculated cross-sectional area by the velocity-time integral value of the left ventricular outflow tract blood flow. In addition, the heart rate can be calculated from a period of a repeating waveform in the Doppler waveform image signal acquired by the Doppler waveform acquisition unit 54, and the cardiac output can be calculated by multiplying the calculated heart rate by the stroke volume.

The determination unit 29 determines whether or not the first measurement value acquired by the first measurement unit 26 and the second measurement value acquired by the second measurement unit 28 are within a predetermined first allowable range and a predetermined second allowable range, respectively. For example, the first allowable range for the first measurement value that is the path length of the left ventricular outflow tract can be set to a range of 1.5 cm or more and 2.5 cm or less. For example, the second allowable range for the second measurement value that is the velocity-time integral value of the left ventricular outflow tract blood flow can be set to a range of 16.2 cm or more and 24.2 cm or less. The first allowable range and the second allowable range are set to numerical ranges in which the first measurement value and the second measurement value can be determined to be normal values, for example, from a clinical viewpoint.

Here, in a case in which the measurement error occurs in the path length of the left ventricular outflow tract or the velocity-time integral value of the left ventricular outflow tract blood flow due to the measurement line ML1 for measuring the left ventricular outflow tract not being disposed at an appropriate position, the position of the Doppler gate G not being disposed at an appropriate position, or the like, and the left ventricular outflow tract or the velocity-time integral value of the left ventricular outflow tract blood flow deviates from the range of the normal value, the stroke volume and the cardiac output may not be accurately calculated. In a case in which the first measurement value is within the first allowable range and the second measurement value is within the second allowable range, it can be determined that the stroke volume and the cardiac output can be accurately calculated.

The notification unit 30 notifies the user of the determination result of the determination unit 29. For example, as shown in FIG. 10, the notification unit 30 can notify the user by displaying a message M representing the determination result on the monitor 23.

In a case in which the first measurement value that is the path length of the left ventricular outflow tract is out of the first allowable range, the notification unit 30 can recommend the user to display the ultrasound image U1 representing the parasternal left ventricular long-axis tomographic plane. In general, it may be easier and more accurate to dispose the measurement line ML1 for measuring the path length of the left ventricular outflow tract in the ultrasound image U1 representing the parasternal left ventricular long-axis tomographic plane than to dispose the measurement line ML1 in the ultrasound image U2 representing the apical five-chamber tomographic plane or the ultrasound image U3 representing the apical three-chamber tomographic plane. In a case in which the ultrasound image U1 representing the parasternal left ventricular long-axis tomographic plane has already been displayed, the notification unit 30 can recommend the user to adjust the position or the posture of the ultrasound probe 1 to redisplay the ultrasound image U1 representing the parasternal left ventricular long-axis tomographic plane.

In a case in which the second measurement value that is the velocity-time integral value of the left ventricular outflow tract blood flow is out of the second allowable range, the notification unit 30 can recommend the user to display the ultrasound image U2 representing the apical five-chamber tomographic plane or the ultrasound image U3 representing the apical three-chamber tomographic plane. In general, it may be easier and more accurate to dispose the Doppler gate G in the ultrasound image U2 representing the apical five-chamber tomographic plane or the ultrasound image U3 representing the apical three-chamber tomographic plane than to dispose the Doppler gate G in the ultrasound image U1 representing the parasternal left ventricular long-axis tomographic plane. In a case in which one of the ultrasound image U2 representing the apical five-chamber tomographic plane or the ultrasound image U3 representing the apical three-chamber tomographic plane has already been displayed, the notification unit 30 can recommend the user to display the ultrasound image representing the other tomographic plane.

The user can appropriately display the ultrasound image and accurately measure the first measurement value that is the path length of the left ventricular outflow tract and the second measurement value that is the velocity-time integral value of the left ventricular outflow tract blood flow by adjusting the position or the posture of the ultrasound probe 1 by checking the determination result notified by the notification unit 30.

The display control unit 22 performs predetermined processing on the ultrasound image acquired by the image acquisition unit 33 and the content of the notification by the notification unit 30 under the control of the apparatus control unit 31, and displays the ultrasound image on the monitor 23.

The monitor 23 displays the ultrasound image and the content of the notification by the notification unit 30 under the control of the display control unit 22, and includes, for example, a display device such as a liquid crystal display (LCD) or an organic electroluminescence display (organic EL display).

The input device 32 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing in the processor 34 is executed by any computer. Moreover, any computer may execute these processes by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor 34 may be composed of one or a plurality of pieces of hardware, and types of hardware are not limited. For example, the processor 34 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). In addition, the types of hardware may be a combination of different types of hardware. In a case in which the plurality of types of hardware are configured to execute one or a plurality of types of processing of a certain processor, the plurality of types of hardware may exist in devices physically separated from each other or may exist in the same device. In addition, in any of the embodiments, the order of each processing by the processor 34 is not limited to the above order, and may be appropriately changed. The hardware is composed of an electric circuit (circuitry) in which circuit elements such as semiconductor elements are combined.

Further, the program may be software, such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

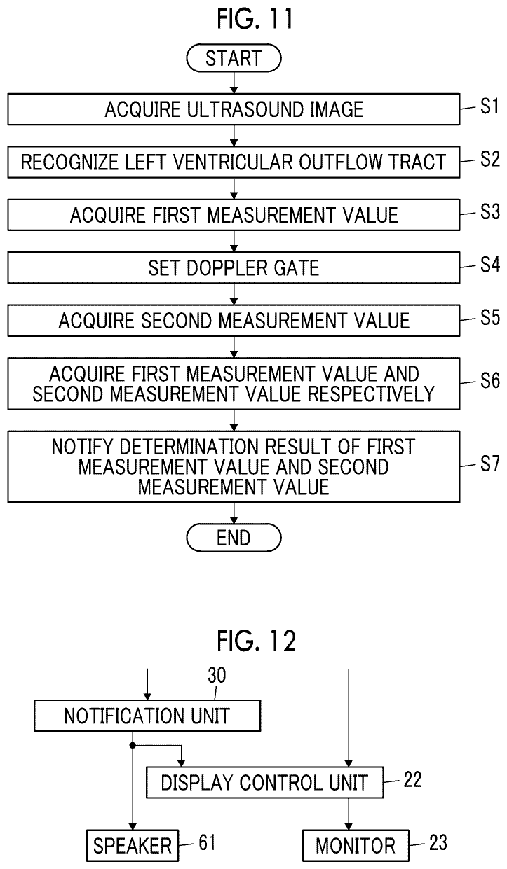

Next, an operation of the ultrasound diagnostic apparatus according to the embodiment will be described with reference to a flowchart shown in FIG. 11. Here, an example will be described in which the image acquisition unit 33 acquires the ultrasound image U1 representing the parasternal left ventricular long-axis tomographic plane.

In step S1, the image acquisition unit 33 acquires the ultrasound image U1 representing the parasternal left ventricular long-axis tomographic plane. In this case, under the control of the apparatus control unit 31, the transmission and reception of the ultrasound from the plurality of transducers of the transducer array 11 are started in accordance with the drive signal from the pulsar 41 of the transmission and reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus body 2, and thus the ultrasound image is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing such as gradation processing. The ultrasound image U1 generated in step S1 in this way is transmitted to the display control unit 22, the image memory 24, and the image recognition unit 25.

In step S2, the image recognition unit 25 recognizes the left ventricular outflow tract T displayed on the parasternal left ventricular long-axis tomographic plane from the ultrasound image U1 acquired by the image acquisition unit 33. The image recognition unit 25 can recognize the left ventricular outflow tract T by, for example, a template matching method, a method of using a trained model in machine learning, or the like.

In step S3, the first measurement unit 26 acquires the diameter of the left ventricular outflow tract T recognized in step S2, that is, the path length of left ventricular outflow tract, as the first measurement value. The first measurement unit 26 can measure, for example, as shown in FIG. 7, a path length of a measurement line ML1 disposed on the left ventricular outflow tract T as the left ventricular outflow tract.

In step S4, the gate setting unit 27 sets the Doppler gate G on the left ventricular outflow tract T recognized in step S2, for example, as shown in FIG. 8.

In step S5, the second measurement unit 28 acquires the velocity-time integral value of the blood flow, that is, the left ventricular outflow tract blood flow in the left ventricular outflow tract T as the second measurement value by measuring the blood flow velocity based on the Doppler frequency shift at the position of the Doppler gate G set in step S4.

In this case, the transceiver circuit 12 controls the transducer array 11 to transmit the ultrasonic wave in a pulsed manner into the subject. The second measurement unit 28 mixes the acoustic signal received from the transceiver circuit 12 with a carrier signal of a reference frequency to orthogonally detect the acoustic signal and convert the acoustic signal into a complex signal, removes a frequency component derived from the movement of the tissue inside the body of the subject from the complex signal, performs frequency analysis by performing Fourier transform on the complex signal of a plurality of sample points to obtain the blood flow velocity, and generates a spectrum signal. The second measurement unit 28 further acquires the Doppler waveform image signal by representing the magnitude of each frequency component with brightness while aligning the spectrum signal on the time axis, specifies a cardiac cycle period in the Doppler waveform image signal, and calculates the velocity-time integral value by time-integrating the value of the flow velocity in the Doppler waveform image signal for the cardiac cycle period. The second measurement unit 28 can specify a repetition unit of the waveform in the Doppler waveform image signal by analyzing the Doppler waveform image signal, and can specify a period to which the specified repetition unit belongs as the cardiac cycle period.

In step S6, the determination unit 29 determines whether or not the first measurement value acquired in step S3 and the second measurement value acquired in step S5 are within the predetermined first allowable range and the predetermined second allowable range, respectively. The determination unit 29 can set, for example, the first allowable range for the first measurement value that is the path length of the left ventricular outflow tract to a range of 1.5 cm or more and 2.5 cm or less, and set the second allowable range for the second measurement value that is the velocity-time integral value of the left ventricular outflow tract blood flow to a range of 16.2 cm or more and 24.2 cm or less. The first allowable range and the second allowable range are set to numerical ranges in which the first measurement value and the second measurement value can be determined to be normal values, for example, from a clinical viewpoint.

In step S7, the notification unit 30 notifies the user of the determination result in step S6. For example, as shown in FIG. 10, the notification unit 30 can notify the user by displaying a message M representing the determination result on the monitor 23.

In this case, in a case in which the first measurement value that is the path length of the left ventricular outflow tract is out of the first allowable range, the notification unit 30 can recommend the user to display the ultrasound image U1 representing the parasternal left ventricular long-axis tomographic plane. In general, it may be easier and more accurate to dispose the measurement line ML1 for measuring the path length of the left ventricular outflow tract in the ultrasound image U1 representing the parasternal left ventricular long-axis tomographic plane than to dispose the measurement line ML1 in the ultrasound image U2 representing the apical five-chamber tomographic plane or the ultrasound image U3 representing the apical three-chamber tomographic plane. In a case in which the ultrasound image U1 representing the parasternal left ventricular long-axis tomographic plane has already been displayed, the notification unit 30 can recommend the user to adjust the position or the posture of the ultrasound probe 1 to redisplay the ultrasound image U1 representing the parasternal left ventricular long-axis tomographic plane.

In addition, in a case in which the second measurement value that is the velocity-time integral value of the left ventricular outflow tract blood flow is out of the second allowable range, the notification unit 30 can recommend the user to display the ultrasound image U2 representing the apical five-chamber tomographic plane or the ultrasound image U3 representing the apical three-chamber tomographic plane. In general, it may be easier and more accurate to dispose the Doppler gate G in the ultrasound image U2 representing the apical five-chamber tomographic plane or the ultrasound image U3 representing the apical three-chamber tomographic plane than to dispose the Doppler gate G in the ultrasound image U1 representing the parasternal left ventricular long-axis tomographic plane. In a case in which one of the ultrasound image U2 representing the apical five-chamber tomographic plane or the ultrasound image U3 representing the apical three-chamber tomographic plane has already been displayed, the notification unit 30 can recommend the user to display the ultrasound image representing the other tomographic plane.

In a case in which the user checks the determination result in step S6 in step S7 and discovers that at least one of the first measurement value or the second measurement value is an abnormal value, the user can adjust the position and the posture of the ultrasound probe 1 such that the value is a normal value. As a result, the user can appropriately display the ultrasound image and accurately measure the first measurement value that is the path length of the left ventricular outflow tract and the second measurement value that is the velocity-time integral value of the left ventricular outflow tract blood flow.

In a case in which the processing of step S7 is completed in this manner, the operation of the ultrasound diagnostic apparatus according to the flowchart of FIG. 11 is completed. Thereafter, for example, the stroke volume or the cardiac output can be accurately calculated by using the first measurement value acquired in step S3 and the second measurement value acquired in step S5.

As described above, according to the ultrasound diagnostic apparatus of the embodiment of the present invention, the first measurement unit 26 acquires the path length of the left ventricular outflow tract as the first measurement value, the second measurement unit 28 acquires the velocity-time integral value of the left ventricular outflow tract blood flow as the second measurement value by measuring the blood flow velocity based on the Doppler frequency shift at the position of the Doppler gate G, the determination unit 29 determines whether or not the first measurement value and the second measurement value are within the predetermined first allowable range and the predetermined second allowable range, respectively, and the notification unit 30 notifies the user of the determination result of the determination unit 29, so that the left ventricular outflow tract and the velocity-time integral value of the left ventricular outflow tract blood flow can be accurately measured.

Note that the description has been made in which the transmission and reception circuit 12 is included in the ultrasound probe 1, but the transmission and reception circuit 12 may be included in the apparatus main body 2.

In addition, the description has been made in which the image generation unit 21 is included in the apparatus main body 2, but the image generation unit 21 may be included in the ultrasound probe 1.

The apparatus main body 2 may be a so-called stationary type, a portable type that is easy to carry, or a so-called handheld type that is configured by, for example, a smartphone or a tablet type computer. As described above, the type of the device constituting the apparatus main body 2 is not particularly limited.

Although an example has been described in which the notification unit 30 notifies the user of the determination result of the determination unit 29 by displaying the determination result on the monitor 23, a method of the notification by the notification unit 30 is not particularly limited thereto. For example, in a case in which the ultrasound diagnostic apparatus includes a speaker 61 as shown in FIG. 12, the notification unit 30 can also output the determination result of the determination unit 29 from the speaker 61 in a voice.

The determination unit 29 can determine that the first measurement value is out of the first allowable range in a case in which an angle range in which an angle between the measurement line ML1 in a case in which the first measurement unit 26 measures the path length of the left ventricular outflow tract as the first measurement value and the scanning line EL passing through the center point of the Doppler gate G is determined, for example, is an angle range of 80 degrees to 90 degrees in a case in which the ultrasound image U1 represents the parasternal longitudinal axis section. In general, the Doppler gate G obtains a value of a component of the original blood flow velocity in a direction orthogonal to the measurement direction, that is, the scanning line EL passing through the center point of the Doppler gate G, so that a value of the component obtained in a case in which the angle between the measurement line ML1 and the scanning line EL passing through the center point of the Doppler gate G is in the determined angle range is extremely small, and an accurate value may not be obtained as the first measurement value.

As a result of the user adjusting the position and the posture of the ultrasound probe 1, a direction orthogonal to the measurement direction of the blood flow by the Doppler gate G, that is, the scanning line EL passing through the center point of the Doppler gate G may deviate from a direction of the blood flow to be measured, that is, a direction of the left ventricular outflow tract T. The Doppler gate G obtains a value of a component of the original blood flow velocity in a direction along the measurement direction, that is, the scanning line EL passing through the center point of the Doppler gate G, but in a case in which the direction along the scanning line EL deviates from a direction orthogonal to the direction of the left ventricular outflow tract T, a difference between the measured value of the blood flow velocity and the original value is increased.

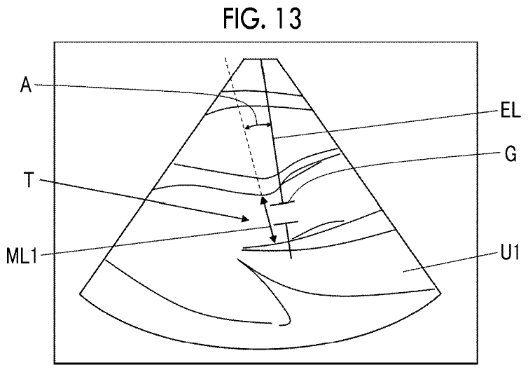

Since the direction along the measurement line ML1 can be regarded as being orthogonal to the direction of the left ventricular outflow tract T, the second measurement unit 28 can correct the blood flow velocity based on an angle A between the measurement line ML1 and the scanning line EL passing through the center point of the Doppler gate G, for example, as shown in FIG. 13, and calculate the velocity-time integral value by using the corrected blood flow velocity. In a case in which the velocity-time integral value calculated based on the Doppler gate G is MV1 and the corrected velocity-time integral value is MV2, there is a simple relationship of MV2=MV1×cos(A), so that the corrected velocity-time integral value can be calculated by MV1=MV2/cos(A). Here, cos is a cosine function.

### EXPLANATION OF REFERENCES

1: ultrasound probe
2: apparatus main body
11: transducer array
12: transmission and reception circuit
21: image generation unit
22: display control unit
23: monitor
24: image memory
25: image recognition unit
26: first measurement unit
27: gate setting unit
28: second measurement unit
29: determination unit
30: notification unit
31: apparatus control unit
32: input device
33: image acquisition unit
34: processor
41: pulsar
42: amplifying unit
43: AD conversion unit
44: beam former
45: signal processing unit
46: DSC
47: image processing unit
51: quadrature detection unit
52: high-pass filter
53: fast Fourier transformation unit
54: Doppler waveform acquisition unit
55: integration value calculation unit
61: speaker
A: angle
EL: scanning line
G: Doppler gate
M: message
ML1: measurement line
T: left ventricular outflow tract
U1, U2, U3: ultrasound image

What is claimed is:

1. An ultrasound diagnostic apparatus comprising:
a processor configured to:
acquire an ultrasound image in which a predetermined cross section of a heart of a subject is imaged;
recognize, from the ultrasound image, a left ventricular outflow tract depicted in the cross section;
acquire, as a first measurement value, a path length of the left ventricular outflow tract;
set a Doppler gate in the left ventricular outflow tract;
acquire, as a second measurement value, a velocity time integral value of blood flow in the left ventricular outflow tract by measuring a blood flow velocity based on a Doppler frequency shift at a position of the Doppler gate;
determine whether the first measurement value and the second measurement value are respectively within a predetermined first allowable range and a predetermined second allowable range; and
notify a user of a determination result of the first measurement value and the second measurement value.

2. The ultrasound diagnostic apparatus according to claim 1,
wherein the processor is configured to acquire the ultrasound image in which, as the predetermined cross section, a parasternal longitudinal axis section of a left ventricle is imaged.

3. The ultrasound diagnostic apparatus according to claim 2,
wherein the processor is configured to:
upon determining that the first measurement value is out of the first allowable range, notify a proposal to reacquire the ultrasound image for the parasternal longitudinal axis section of the left ventricle; and
upon determining that the second measurement value is out of the second allowable range, notify a proposal to reacquire the ultrasound image using, as the predetermined cross section, an apical three-chamber section or an apical five-chamber section.

4. The ultrasound diagnostic apparatus according to claim 1,
wherein the processor is configured to acquire the ultrasound image in which, as the predetermined cross section, an apical three-chamber section is imaged.

5. The ultrasound diagnostic apparatus according to claim 4,
wherein the processor is configured to:
upon determining that the first measured value is out of the first allowable range, notify a proposal to reacquire the ultrasound image using, as the predetermined cross section, the parasternal longitudinal axis section of the left ventricle; and
upon determining that the second measurement value is out of the second allowable range, notify a proposal to reacquire the ultrasound image, as the predetermined cross section, an apical three-chamber section or notify a proposal to reacquire the ultrasound image using, as the predetermined cross section, an apical five-chamber section.

6. The ultrasound diagnostic apparatus according to claim 1,
wherein the processor is configured to acquire the ultrasound image in which, as the predetermined cross section, an apical five-chamber section is imaged.

7. The ultrasound diagnostic apparatus according to claim 6,
wherein the processor is configured to:
upon determining that the first measurement value is out of the first allowable range, notify a proposal to reacquire the ultrasound image using, as the predetermined cross section, the parasternal longitudinal axis section of the left ventricle; and
upon determining that the second measurement value is out of the second allowable range, notify a proposal to reacquire the ultrasound image, as the predetermined cross section, an apical five-chamber section or notify a proposal to reacquire the ultrasound image using, as the predetermined cross section, an apical three-chamber section.

8. The ultrasound diagnostic apparatus according to claim 1, further comprising:
a monitor,
wherein the processor is configured to display the determination result.

9. The ultrasound diagnostic apparatus according to claim 2, further comprising:
a monitor,
wherein the processor is configured to display the determination result by the determination unit on the monitor.

10. The ultrasound diagnostic apparatus according to claim 4, further comprising:
a monitor,
wherein the processor is configured to display the determination result by the determination unit on the monitor.

11. The ultrasound diagnostic apparatus according to claim 6, further comprising:
a monitor,
wherein the processor is configured to display the determination result by the determination unit on the monitor.

12. The ultrasound diagnostic apparatus according to claim 1, further comprising:
a speaker,
wherein the processor is configured to output the determination result as sound from the speaker.

13. The ultrasound diagnostic apparatus according to claim 2, further comprising:
a speaker,
wherein the processor is configured to output the determination result as sound from the speaker.

14. The ultrasound diagnostic apparatus according to claim 4, further comprising:
a speaker,
wherein the processor is configured to output the determination result as sound from the speaker.

15. The ultrasound diagnostic apparatus according to claim 6, further comprising:
a speaker,
wherein the processor is configured to output the determination result as sound from the speaker.

16. The ultrasound diagnostic apparatus according to claim 1,
wherein the processor is further configured to determine that the first measurement value is out of the first allowable range upon determining that an angle formed between a measurement line used in measuring the path length of the left ventricular outflow tract as the first measurement value and a scan line passing through a center point of the Doppler gate is within a predetermined angle range.

17. The ultrasound diagnostic apparatus according to claim 2,
wherein the processor is further configured to determine that the first measurement value is out of the first allowable range upon determining that an angle formed between a measurement line used in measuring the path length of the left ventricular outflow tract as the first measurement value and a scan line passing through a center point of the Doppler gate is within a predetermined angle range.

18. The ultrasound diagnostic apparatus according to claim 4,
wherein the processor is further configured to determine that the first measurement value is out of the first allowable range upon determining that an angle formed between a measurement line used in measuring the path length of the left ventricular outflow tract as the first measurement value and a scan line passing through a center point of the Doppler gate is within a predetermined angle range.

19. The ultrasound diagnostic apparatus according to claim 1,
wherein the processor is configured to:
correct the blood flow velocity based on an angle formed between a measurement line used in measuring the path length of the left ventricular outflow tract as the first measurement value and a scan line passing through a center point of the Doppler gate; and
calculate the velocity time integral value using the corrected blood flow velocity.

20. A control method of an ultrasound diagnostic apparatus, the method comprising:
acquiring an ultrasound image in which a predetermined cross section of a heart of a subject is imaged;
recognizing, from the acquired ultrasound image, a left ventricular outflow tract depicted in the cross section;
acquiring, as a first measurement value, a path length of the recognized left ventricular outflow tract;
setting a Doppler gate in the recognized left ventricular outflow tract;
acquiring, as a second measurement value, a velocity time integral value of blood flow in the left ventricular outflow tract by measuring a blood flow velocity based on a Doppler frequency shift at a position of the set Doppler gate;
determining whether the acquired first measurement value and the acquired second measurement value are respectively within a predetermined first allowable range and a predetermined second allowable range; and
notifying a user of a determination result of the first measurement value and the second measurement value.
